(19) United States
(12) Patent Application Publication
(10) Pub. No.: US 2006/0292207 A1
(43) Pub. Date: Dec. 28, 2006

(54) Chitosan based dressing

(75) Inventors: Te-Hsing Wu, Taoyuan City (TW); Jen-Ming Yang, Gueishan Township (TW); Ying-Kai Fu, Longtan Township (TW); Hao-Tzu Lin, Bade City (TW); Chia-Chieh Chen, Longtan Township (TW)

Correspondence Address: TROXELL LAW OFFICE PLLC, SUITE 1404, 5205 LEESBURG PIKE, FALLS CHURCH, VA, 22041, US

(73) Assignee: Atomic Energy Council - Institute of Nuclear Energy Research

(21) Appl. No.: 11/158,058

(22) Filed: Jun. 22, 2005

(51) Int. Cl. A61L 15/00; A61K 31/722

(52) U.S. Cl. 424/445; 514/55

(57) ABSTRACT

The present invention is a dressing capable of hydrotaxis and temperature-sensitivity for a wound or for antisepsis, containing layers of acrylic acid, N-isopropyl acrylamide and chitosan by a grafting through an ionizing radiation, a UV radiation, a peracid process and a freeze-drying process.

### FIELD OF THE INVENTION

The present invention relates to a dressing; more particularly, relates to acrylic acid, N-isopropyl acrylamide (NIPAAm) and chitosan grafting with and fixing on the substrate to obtain a dressing for a wound or for antisepsis.

### DESCRIPTIONS OF THE RELATED ARTS

A graft polymerization by a gamma-ray radiation or a UV radiation is widely applied in the plastic industry. Such as, H. T. Lokhande etc. reported in 1993 that acrylic nitrile was used to be grafted with plastic by a gamma-ray radiation (H. T. Lokhande etc., J. of Appl. Poly. Sci. 48,495, 1993). A prior art of “Preperation of easily stripped off temporary wound dressing materials by radiation grafting”, No. 458784, patent of Taiwan, uses a nonwoven fabric processed through a gamma-ray radiation for a graft polymerization on surface. Cobalt-60 (Co-60) is used to radiate a gamma-ray to a nonwoven fabric to obtain a radical or a peroxide to be grafted together with NIPAAm monomer on the nonwoven fabric. A bare nonwoven fabric has no disinfection ability except helping germs grow. After obtaining acrylic acid and NIPAAm, a disinfection ability is obtained yet lacking of enough effectiveness. Furthermore, a layer of NIPAAm forms a dense layer hindering water vapor from transmitting so that the water vapor transmission rate, the ventilation and the gas permeability is reduced into disappointment. So, the prior arts do not fulfill users' requests on actual use

### SUMMARY OF THE INVENTION

The main purpose of the present invention is to provide a dressing with chitosan having antisepsis, hydrotaxis and temperature-sensitivity.

To achieve the above purpose, the present invention is a dressing with chitosan. By an ionizing radiation or a UV radiation, acrylic acid, or acrylic acid together with NIPAAm, is grafted and fixed on a surface of a substrate to obtain a layer of acrylic acid or acrylic acid together with NIPAAm. Then, chitosan is grafted and fixed on the layer of acrylic acid or acrylic acid together with NIPAAm by an ionizing radiation, a UV radiation, or a freeze-drying process to obtain a layer of chitosan. The chitosan is grafted into a disordered copolymer or a dual-grafted polymer. With the thicker depth, its structure is dense and porous. In the end, a material for disinfection (such as Ag, Zn and Al) is sprayed over the dressing to improve antisepsis ability. Accordingly, a dressing with chitosan having antisepsis, hydrotaxis and temperature-sensitivity is obtained for a wound or for antisepsis.

### BRIEF DESCRIPTIONS OF THE DRAWINGS

The present invention will be better understood from the following detailed descriptions of the preferred embodiments according to this invention, taken in con junction with the accompanying drawings, in which

FIG. 1A is a cross-sectional view of a dressing with chitosan according to a first embodiment of the present invention;

FIG. 1B is a cross-sectional view of a dressing with chitosan according to a second embodiment of the present invention;

FIG. 2A is a cross-sectional view of a dressing AN10C according to the first embodiment of the present invention;

FIG. 2B is a SEM (scanning electron microscope) cross-sectional view of a nonwoven fabric before being processed according to the present invention;

FIG. 3A is a SEM cross-sectional view of the dressing AN10C at a magnifying power of 150 according to the first embodiment of the present invention;

FIG. 3B is a SEM cross-sectional view of the dressing AN10C at a magnifying power of 600 according to the first embodiment of the present invention;

FIG. 4A is a SEM cross-sectional view of a dressing AN11C at a magnifying power of 150 according to the first embodiment of the present invention;

FIG. 4B is a SEM cross-sectional view of the dressing AN11C at a magnifying power of 600 according to the first embodiment of the present invention;

FIG. 5A is a SEM cross-sectional view of a dressing PP-nipga-chio at a magnifying power of 40 according to the second embodiment of the present invention;

FIG. 5B is a SEM cross-sectional view of the dressing PP-nipga-chio at a magnifying power of 150 according to the second embodiment of the present invention;

FIG. 6A is a SEM cross-sectional view of a dressing PP-nipga-chiN at a magnifying power of 40 according to the second embodiment of the present invention

FIG. 6B is a SEM cross-sectional view of the dressing PP-nipga-chiN at a magnifying power of 150 according to the second embodiment of the present invention;

FIG. 7 is a view showing absorbing times concerning thermo-sensitivity for dressings according to the first embodiment of the present invention;

FIG. 8 is a view showing effects of disinfection for dressings according to the first embodiment of the present invention;

FIG. 9 is a view showing water vapor transmission rates for dressings according to the first embodiment of the present invention;

FIG. 10 is a view showing absorbing times for dressing's according to the second embodiment of the present invention;

FIG. 11 is a view showing absorbing times concerning thermo-sensitivity for dressings according to the second embodiment of the present invention;

FIG. 12 is a view showing disinfection for some dressings according to the second embodiment of the present invention;

FIG. 13 is a view showing water vapor transmission rates for dressings according to the second embodiment of the present invention;

FIG. 14 is a view showing disinfection for a nonwoven fabric with N-isopropyl acrylamide (NIPAAm) according to the present invention;

FIG. 15 is a view showing effects of disinfection according to the present invention.

### DESCRIPTION OF THE PREFERRED EMBODIMENTS

The following descriptions of the preferred embodiments are provided to understand the features and the structures of the present invention.

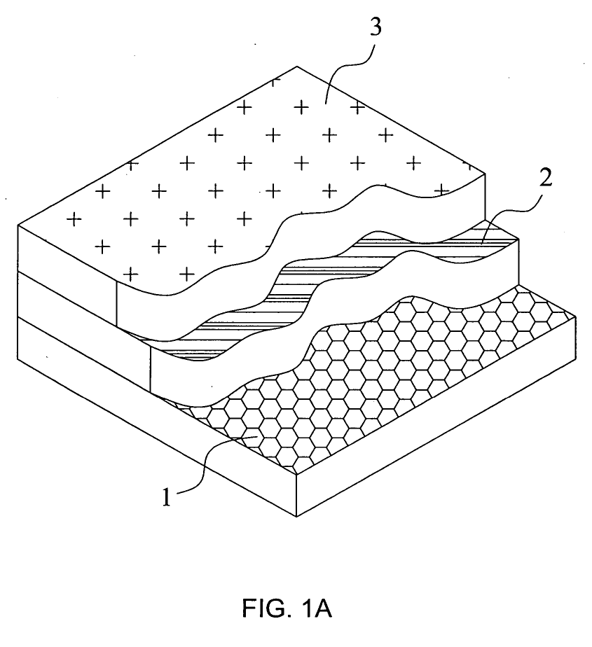

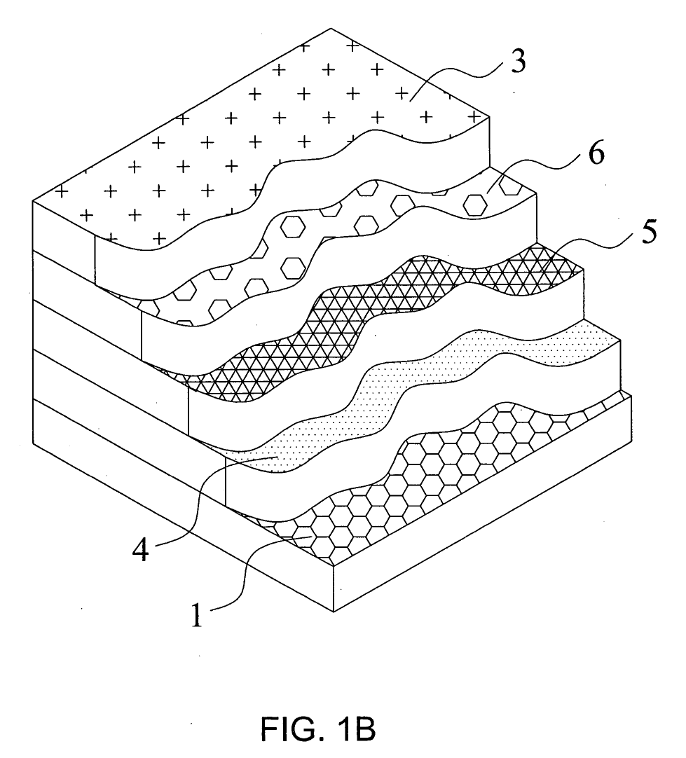

Please refer to FIG. 1A, which is a cross-sectional view of a dressing with chitosan according to a first embodiment of the present invention. As shown in the figures, the present invention is a dressing with chitosan, comprising (a) a substrate [1], (b) a layer of acrylic acid together with NIPAAm [2] fixed on the substrate [1], and (c) a layer of chitosan [3] fixed on the layer of acrylic acid together with NIPAAm [2], sprayed over with a solution of AgNO₃. Therein, the substrate [1] is a nonwoven fabric (such as polyethylene, polypropylene, polyethylene terephthalate, cotton, etc.), a PVC (polyvinyl chloride) fabric, or a fabric made of other film-shaped or flat material (such as macromolecule, cellulose, etc.). The layer of acrylic acid together with NIPAAm [2] is formed by grafting acrylic acid and NIPAAm on the substrate [1] using an ionizing radiation or a UV (ultraviolet) radiation over a mixed solution of acrylic acid and NIPAAm, or other similar monomer, or hydrogel group, having a mixture rate of 1:0, 1:1, or 3:1. The layer of chitosan [3] is formed by grafting chitosan on the layer of acrylic acid together with NIPAAm [2] using an ionizing radiation or a UV radiation over a solution of chitosan [3]. The other monomer is a similar monomer of vinyl monomer, such as acrylic acid, NIPAAm with derivatives thereof, 2-hydroxy ethyl methacrylate, 2-hydroxy ethyl methacrylate with derivatives thereof, vinylpyridine, vinylpyridine with derivatives thereof, etc. And, the hydrogel group is selected from acrylic acid, 2-hydroxy ethyl methacrylate, etc.

In addition, the present invention provides another embodiment of a dressing with chitosan, comprising (a) a substrate [1], (b) a layer of acrylic acid [4] fixed on the substrate [1], (c) a layer of peroxyacid group [5] fixed on the layer of acrylic acid [4], (d) a layer of acrylic acid together with NIPAAm [6] fixed on the layer of peroxyacid group [5], and (e) a layer of chitosan [3] fixed on the layer of acrylic acid together with NIPAAm [6], sprayed over with a solution of AgNO₃. Therein, the layer of acrylic acid [4] is formed by grafting acrylic acid on the substrate [1] through gamma-ray radiation or a UV radiation over a solution of acrylic acid, which uses ethanol as a dissolvent to dissolve 30% (V/V) of acrylic acid or other similar monomer (such as methacrylic acid, 3-butenoic acid, hydrosorbic acid, etc.). After the radiation, dis-grafted monomers of acrylic acid on the layer of acrylic acid [4] is washed away by ethanol and acetone. Then, the layer of peroxyacid group [5] is formed by a peracid process using sulfuric acid and hydrogen peroxide. The sulfuric acid and the hydrogen peroxide left on the layer of peroxyacid group [5] after the process is removed by methanol. The layer of NIPAAm [6] is formed by grafting NIPAAm on the layer of peroxyacid group [5] through an ionizing radiation or a UV radiation. And, then, the layer of chitosan [3] is formed by grafting chitosan on the layer of peroxyacid group [5] through a freeze-drying process under 0° C.˜−196° C. Consequently, a dressing with chitosan is obtained.

For further understanding, the present invention is illustrated with the following examples:

### EXAMPLE 1

### A Nonwoven Fabric with Acrylic Acid and Chitosan Fixed on Through a UV Radiation

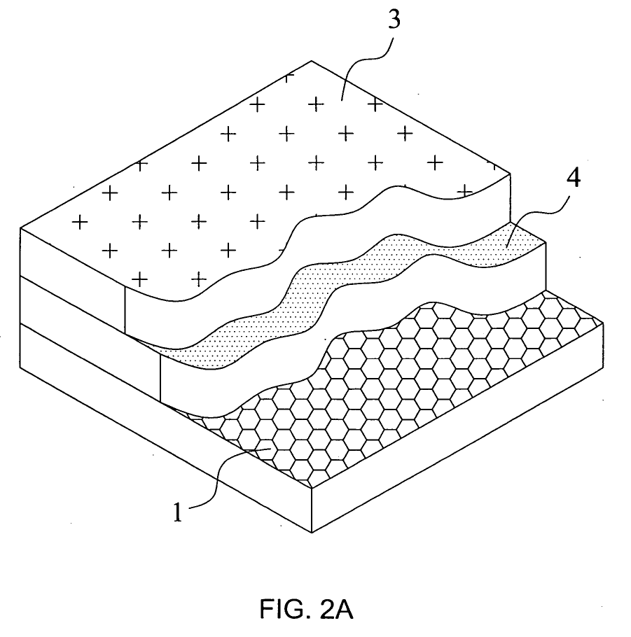

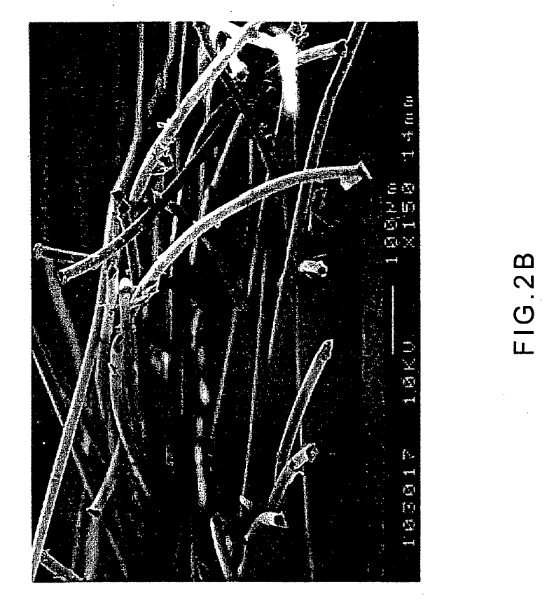

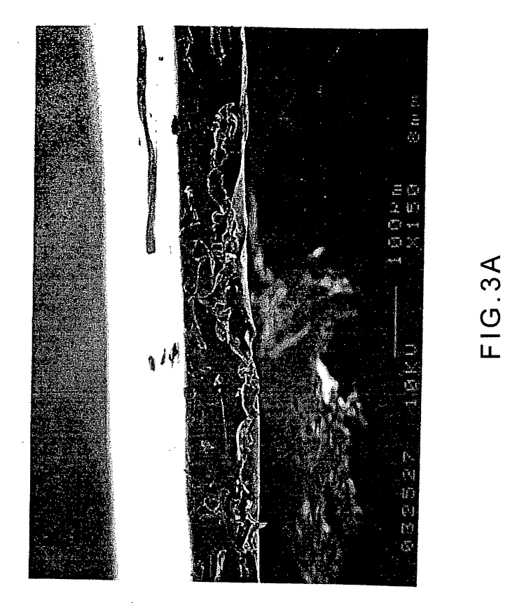

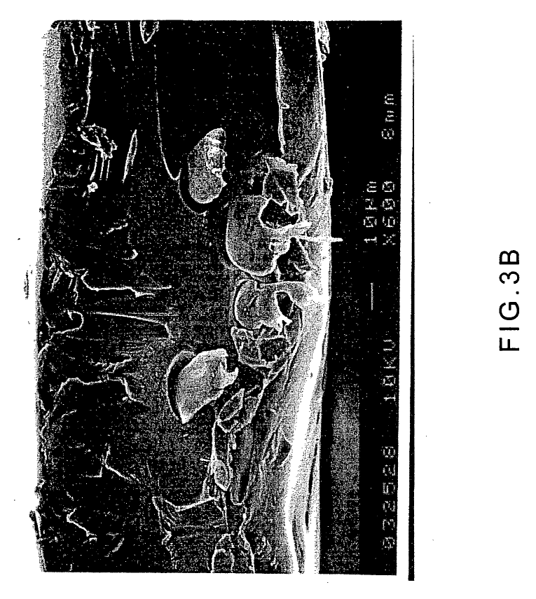

Please refer to FIG. 2A through FIG. 3B, which are a cross-sectional view of a dressing AN10C according to the first embodiment of the present invention, a SEM (scanning electron microscope) cross-sectional view of a nonwoven fabric before being processed according to the present invention, and SEM cross-sectional views of the dressing AN10C at a magnifying power of 150 and another magnifying power of 600. As shown in the figures, the present invention uses a nonwoven fabric as a substrate [1] (8 cm×8 cm); and a solution of acrylic acid with a dissolvent of ethanol having less than 10% (V/V) and less than 5 wt % of benzoin ethyl ether as a photo initiator, so that acrylic acid is grafted on the substrate [1] through a UV radiation for 10˜100 minutes to form a layer of acrylic acid [4]. And by using a solution of chitosan with a 0.1M dissolvent of acetic acid in a thickness of 1˜5 wt % having less than 10% (V/V) and less than 5 wt % of benzoin ethyl ether as a photo initiator, chitosan is fixed on the layer of acrylic acid [4] through a UV radiation for 10˜100 minutes to form a layer of chitosan [3].

### EXAMPLE 2

### A Nonwoven Fabric with Acrylic Acid, NIPAAm and Chitosan Fixed on Through a UV Radiation

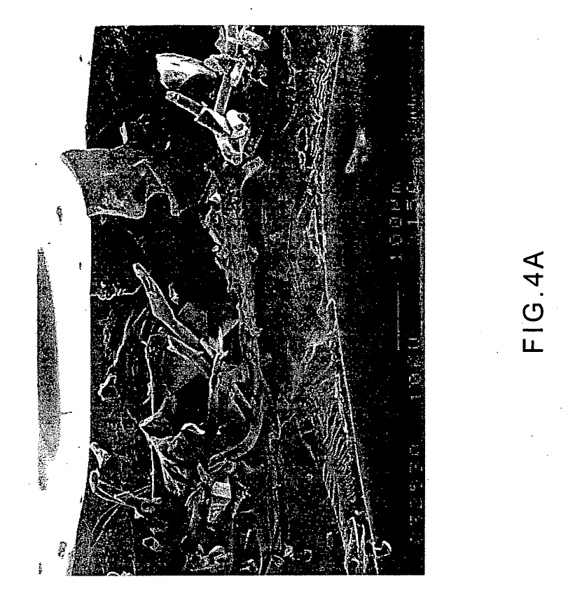

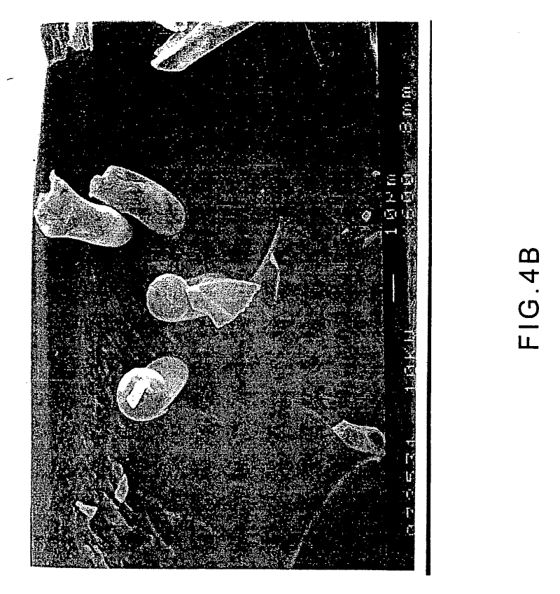

Please refer to FIG. 1A, FIG. 4A and FIG. 4B, which, according to the first embodiment of the present invention, are the cross-sectional view of the dressing with chitosan and SEM cross-sectional views of the dressing AN11C at a magnifying power of 150 and another magnifying power of 600. As shown in the figures, the present invention uses a nonwoven fabric as a substrate [1] (8 cm×8 cm); and a solution of acrylic acid (10% V/V in a dissolvent of ethanol) and NIPAAm (0.148 g NIPAAm/1 ml ethanol), whose mixing rate is 1:1, having less than 10% (V/V) and less than 5 wt % of benzoin ethyl ether as a photo initiator, so that acrylic acid and NIPAAm is grafted on the substrate [1] through a UV radiation for 10˜100 minutes to form a layer of acrylic acid together with NIPAAm [2]. And, by using a solution of chitosan with a 0.1M dissolvent of acetic acid in a thickness of 1˜5 wt % having less than 10% (V/V) and less than 5 wt % of benzoin ethyl ether as a photo initiator, chitosan is fixed on the layer of acrylic acid together with NIPAAm [2] through a UV radiation for 10˜100 minutes to form a layer of chitosan [3].

### EXAMPLE 3

### A Nonwoven Fabric with Acrylic Acid, NIPAAm and Chitosan Fixed on Through a Gamma-Ray Radiation, a UV Radiation, a Peracid Process and a Freeze-Drying Process Under 0° C.

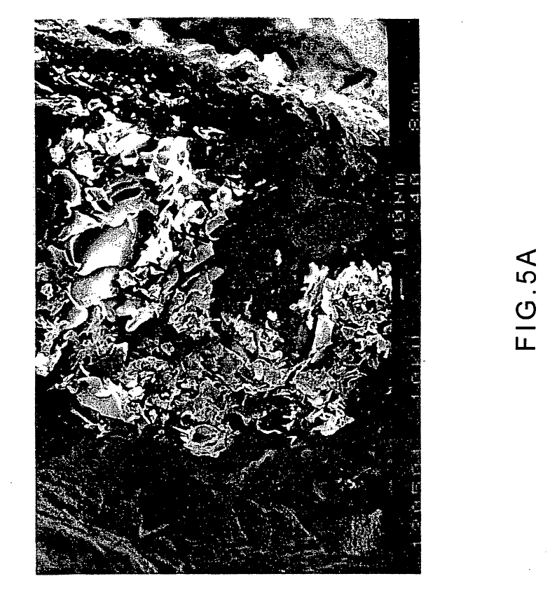

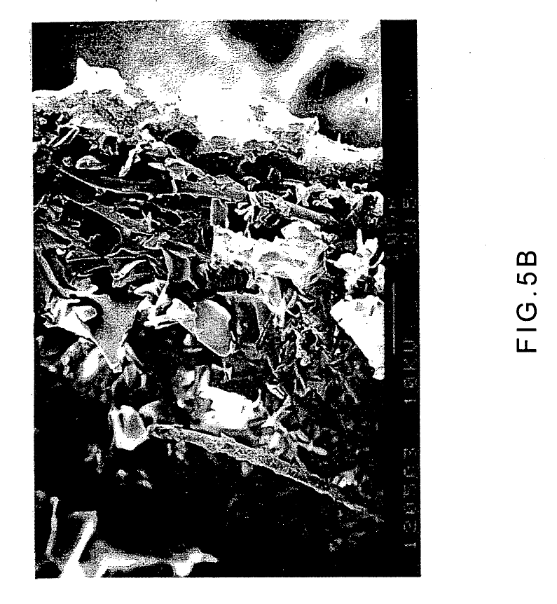

Please refer to FIG. 1B, FIG. 5A and FIG. 5B, which, according to the second embodiment of the present invention, are the cross-sectional view of the dressing with chitosan, and SEM cross-sectional views of the dressing PP-nipga-chio at a magnifying power of 40 and another magnifying power of 150. As shown in the figures, the present invention uses a nonwoven fabric as a substrate [1] (8 cm×8 cm); and a solution of acrylic acid (30% V/V in a dissolvent of ethanol), processed with a gamma-ray radiation of 0.5˜10 KGy/hr for 0.5˜10 hours to a total amount of 5˜60 kGy, so that the acrylic acid is grafted and fixed on the substrate [1] to form a layer of acrylic acid [4]. After the radiation, the layer of acrylic acid [4] is washed by ethanol and acetone. Then, the layer of peroxyacid group [5] is formed by a peracid process using sulfuric acid and hydrogen peroxide under 0° C. for 0.5˜6 hours. The sulfuric acid and hydrogen peroxide left on the layer of peroxyacid group [5] after the process is removed by methanol. Then, the layer of NIPAAm [6] is formed by fixing NIPAAm (in a dissolvent of 0.1M acetic acid) on the layer of peroxyacid group [5] using a UV radiation for 10˜100 minutes. And, then, the layer of chitosan [3] is formed by grafting and fixing chitosan on the layer of NIPAAm [6] using a solution of chitosan with a 0.1M dissolvent of acetic acid in a thickness of 1˜5 wt % having 1 ml of 1 wt % glutaraldehyde for freezing for 24 hours under 0° C. and then through a freeze-drying process under 0° C.

### EXAMPLE 4

### A Nonwoven Fabric with Acrylic Acid, NIPAAm and Chitosan Fixed on Through a Gamma-Ray Radiation, a UV Radiation, a Peracid Process and a Freeze-Drying Process Under −196° C.

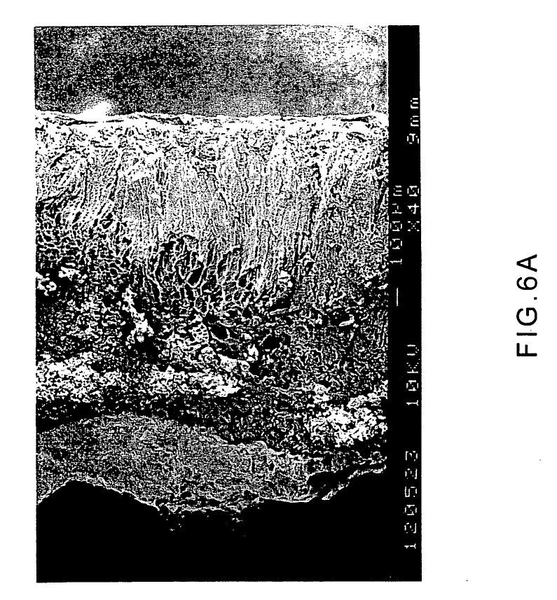

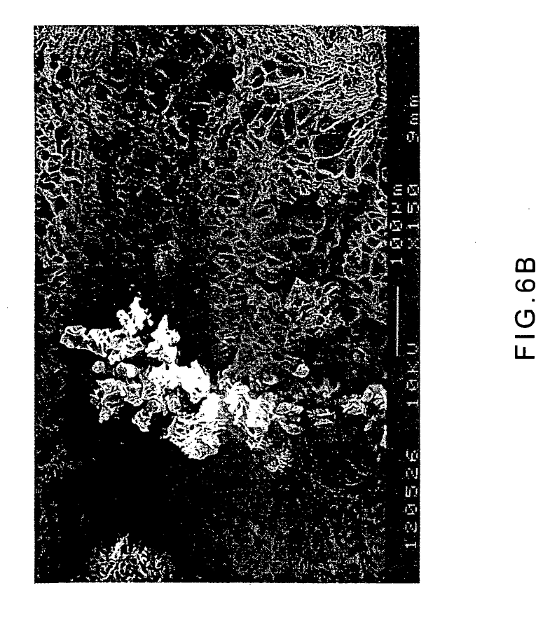

Please refer to FIG. 1B, FIG. 6A and FIG. 6B, which, according to the second embodiment of the present invention, are the cross-sectional view of the dressing with chitosan and SEM cross-sectional views of the dressing PP-nipga-chiN at a magnifying power of 40 and another magnifying power of 150. As shown in the figures, the present invention uses a nonwoven fabric as a substrate [1]; and a solution of acrylic acid (30% V/V in a dissolvent of ethanol), processed with a gamma-ray radiation of 0.5˜10 KGy/hr for 0.5˜10 hours to a total amount of 5˜60 kGy, so that the acrylic acid is grafted and fixed on the substrate [1] to form a layer of acrylic acid [4]. After the radiation, the layer of acrylic acid [4] is washed by ethanol and acetone. Then, the layer of peroxyacid group [5] is formed by a peracid process using sulfuric acid and hydrogen peroxide under 0° C. for 0.5˜6 hours. The sulfuric acid and hydrogen peroxide left on the layer of peroxyacid group [5] after the process is removed by methanol. Then, the layer of NIPAAm [6] is formed by fixing NIPAAm (in a dissolve nt of 0.1M acetic acid) on the layer of peroxyacid group [5] using a UV radiation for 10˜100 minutes. And, then, the layer of chitosan [3] is formed by grafting and fixing chitosan on the layer of NIPAAm [6] using a solution of chitosan with a 0.1M dissolvent of acetic acid in a thickness of 1˜5 wt % having 1 ml of 1 wt % glutaraldehyde for freezing for 24 hours under −196° C. and then through a freeze-drying process under −196° C.

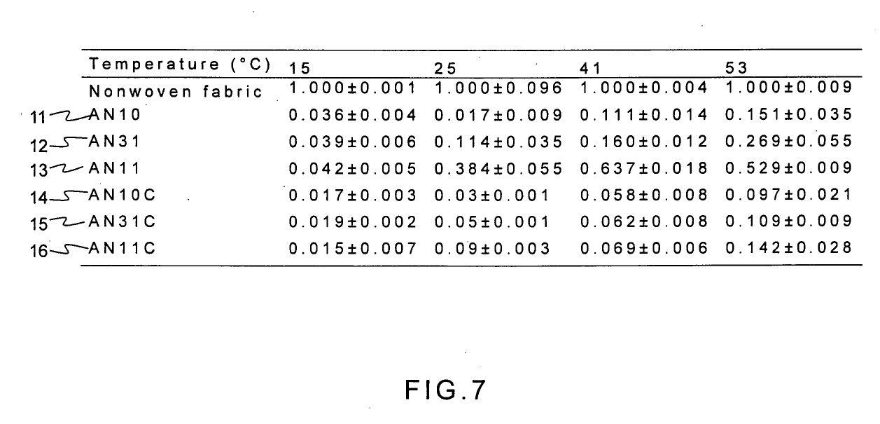

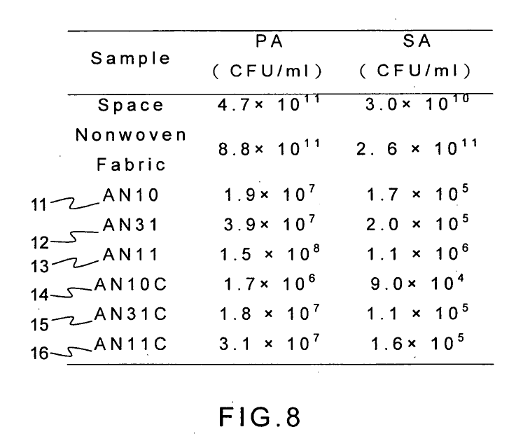

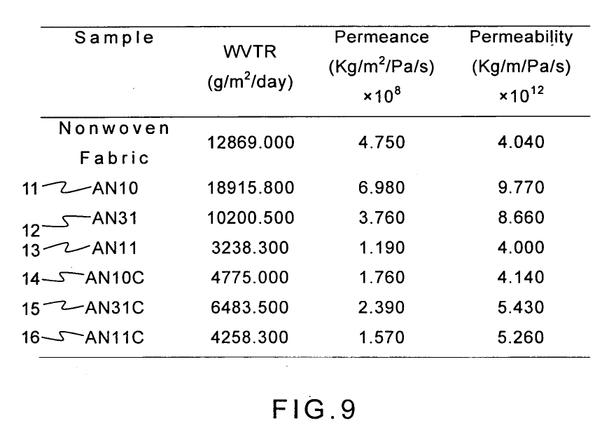

Please refer to FIG. 1A and FIG. 7 through FIG. 9, which, according to the first embodiment of the present invention, are the cross-sectional view of the dressing with chitosan and views for dressings showing absorbing times concerning thermo-sensitivity, effects of disinfection, and water vapor transmission rates. In FIG. 7 through FIG. 9, the CFU is an acronym of Colony Forming Units and the PA is an acronym of Pseudomonas Aeruginosa and SA is an acronym of Staphylococcus Aureus, AN10 [11] is a nonwoven fabric prepared by using a mixed solution of acrylic acid and NIPAAm in a rate of 1:0 processed through a UV radiation; AN31 [12] is a nonwoven fabric prepared by using a mixed solution of acrylic acid and NIPAAm in a rate of 3:1 processed through a UV radiation; AN11 [13] is a nonwoven fabric prepared by using a mixed solution of acrylic acid and NIPAAm in a rate of 1:1 processed through a UV radiation; AN10C [14] is a nonwoven fabric prepared by using AN10 coated with chitosan processed through a UV radiation; AN31C [15] is a nonwoven fabric prepared by using AN31 coated with chitosan processed through a UV radiation; and, AN11C [16] is a nonwoven fabric prepared by using AN11 coated with chitosan processed through a UV radiation. As shown in FIG. 7, the amount of NIPAAm and the absorbing time increase following the reduction of the acrylic acid. Yet, after chitosan is grafted on a layer of acrylic acid together with NIPAAm [2], the absorbing time is reduced out of an increase in hydrotaxis. Concerning the AN31 and the AN11 in FIG. 7, after NIPAAm's are grafted on the nonwoven fabrics, the hydrotaxis at 15° C. and 25° C. is bigger and the hydrotaxis at 41° C. and 53° C. is smaller. It shows that, after the grafting on the nonwoven fabrics, temperature-sensitivity is obtained. Besides, as shown in FIG. 8, after the UV radiation, nonwoven fabrics coated with acrylic acid, NIPAAm and chitosan obtain better disinfection ability. As shown in FIG. 8, bare nonwoven fabric obtains no disinfection ability except helping the germs grow. But, after the nonwoven fabric is coated with acrylic acid and NIPAAm, disinfection ability is obtained to reduce the number of germs. After the chitosan is grafted and fixed on the nonwoven fabric coated with acrylic acid and NIPAAm, better disinfection ability is obtained to reduce a great number of germs. As shown in FIG. 9, the water vapor transmission rate (WVTR), the ventilation and the gas permeability are reduced following the decrease in the acrylic acid and the increase in the NIPAAm, which is owing to the better absorbing ability for the acrylic acid than that for the NIPAAm. So, when the amount of the acrylic acid is decreased, the hydrotaxis and the gas permeability are reduced at the same time. Additionally, after the chitosan is grafted and fixed on the layer of acrylic acid together with NIPAAm [2], the depth and the density are greater to hinder water vapor from transmitting. As a result, the water vapor transmission rate, the ventilation and the gas permeability are reduced.

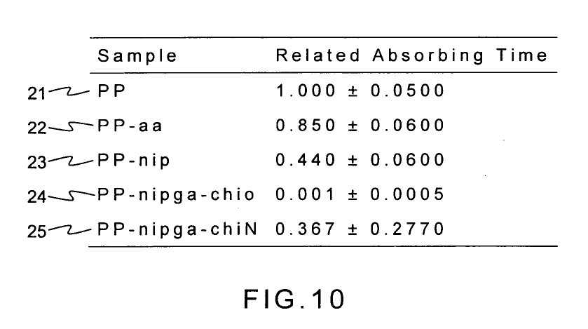

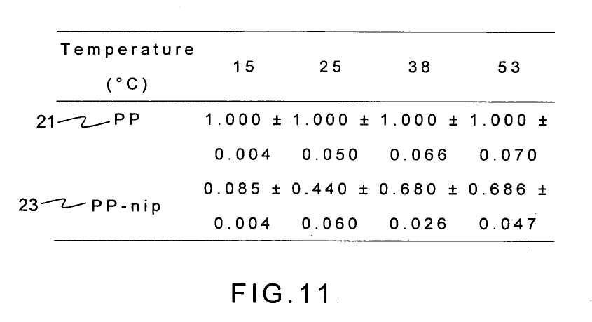

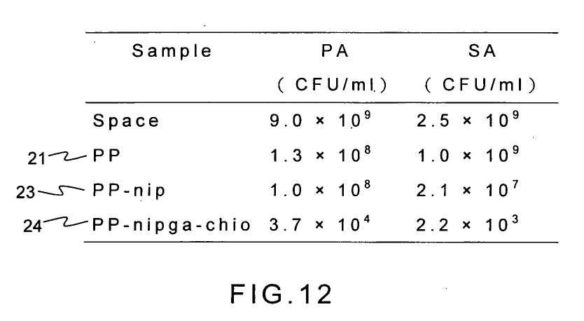

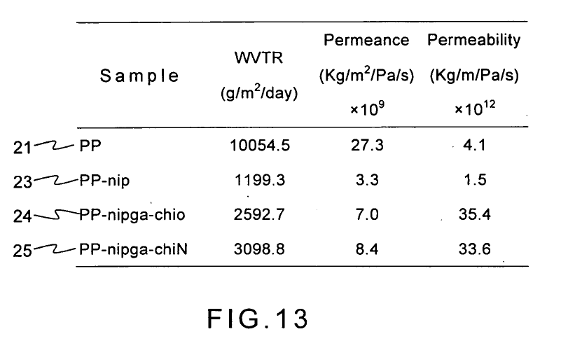

Please refer to FIG. 1A and FIG. 10 through FIG. 13, which, according to the second embodiment of the present invention, are the cross-sectional view of the dressing with chitosan and views for dressings showing absorbing times, thermo-sensitivity, effects of disinfection, and water vapor transmission rates. In FIG. 10 through FIG. 13, the CFU is an acronym of Colony Forming Units and the PA is an acronym of Pseudomonas Aeruginosa and SA is an acronym of Staphylococcus Aureus, PP [21] is a nonwoven fabric; PP-aa [22] is a nonwoven fabric with acrylic acid processed through a gamma-ray radiation; PP-nip [23] is a nonwoven fabric with NIPAAm processed through a gamma-ray radiation, a UV radiation and a peracid process; PP-nipga-chio [24] is a nonwoven fabric with NIPAAm and chitosan processed through a gamma-ray radiation, a UV radiation, a peracid process and a freeze-drying process under 0° C.; PP-nipga-chiN [25] is a nonwoven fabric with NIPAAm and chitosan processed through a gamma-ray radiation, a UV radiation, a peracid process and a freeze-drying process under −196° C.; and, the temperature for the embodiment is 25° C. As shown in FIG. 10, after acrylic acid and NIPAAm is grafted on nonwoven fabrics, the absorbing time is reduced out of an increase in hydrotaxis. After chitosan is coated on, the absorbing time is greatly reduced, which means the hydrotaxis is further increased out of the good hydrotaxis of the chitosan for absorbing a great amount of water in a short time. Hence, after chitosan is coated on, the hydrotaxis of the nonwoven fabric is greatly increased. Moreover, by comparing nonwoven fabric with chitosan made under different temperature, it is found that the dressing with chitosan made under −196° C. obtains better absorbing time. As shown in FIG. 5A through FIG. 6A, the dressing with chitosan made under −196° C. obtains denser structure which hinders water from entering into the dressing. As shown in FIG. 11, after NIPAAm is coated, the absorbing time at 15° C. and 25° C. is smaller than that at 38° C. and 53° C., which means the hydrotaxis at 15° C. and 25° C. is bigger than that at 41° C. and 53° C. It shows that the layer of NIPAAm [6] obtains temperature-sensitivity where the hydrotaxis changes according to temperature. As shown in FIG. 12, a bare nonwoven fabric obtains no disinfection ability except helping the germs grow. But, after the nonwoven fabric is coated with NIPAAm, disinfection ability is getting better. After the chitosan is grafted and fixed on, much better disinfection ability is obtained owing to the disinfection ability of the chitosan. As shown in FIG. 13, after the layer of NIPAAm [6] is obtained, the water vapor transmission rate, the ventilation and the gas permeability is reduced because of hindering the ventilation of the water vapor. After chitosan is grafted and fixed on, the water vapor transmission rate, the ventilation and the gas permeability are improved owing to the better hydrotaxis of chitosan. Moreover, by comparing nonwoven fabric with chitosan made under different temperature, it is found that the dressing with chitosan made under −196° C. obtains better water vapor transmission rate.

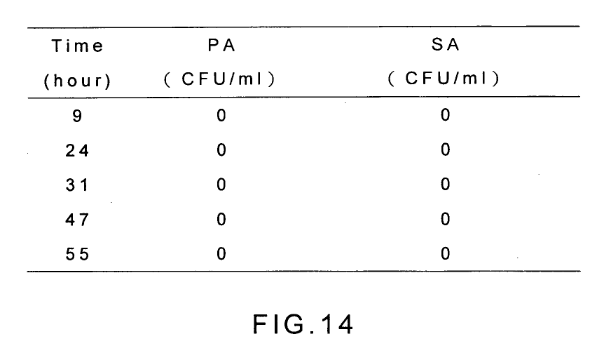

Please refer to FIG. 1B and FIG. 14, which are a cross-sectional view of a dressing with chitosan according to the second embodiment of the present invention and a view showing disinfection for a nonwoven fabric with NIPAAm according to the present invention. As shown in the figure, because each of the dressings with chitosan contains a layer of NIPAAm [6] through a grafting, dressings having the layer of NIPAAm [6] are taken into a test for disinfection. After 55 hours of the test, no germ passes through any of the dressings having the layer of NIPAAm [6], which means a dressing having a layer of NIPAAm obtains good ability in hindering the transmission of germs.

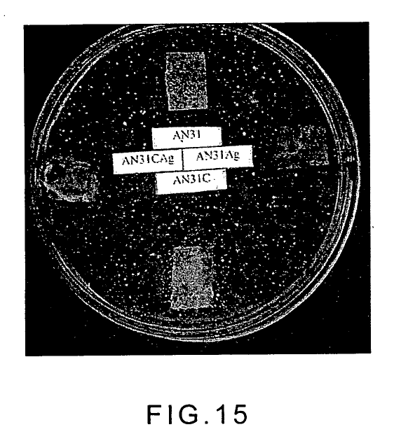

Please refer to FIG. 15, which is a view showing effects of disinfection according to the present invention. As shown in the figure, a dressing with chitosan is sprayed with a solution having metal ions, such as ions of Ag, Zn or Al, to improve antisepsis ability of the dressing. Taking the AN31 (a nonwoven fabric prepared by using a mixed solution of acrylic acid and NIPAAm in a rate of 3:1 processed through a UV radiation) sprayed with a solution of AgNO₃ as an example, a white space (an area with no germ) is shown around the nonwoven fabric, which means a dressing with chitosan according to the present invention is antiseptic. Also shown in the figure, the nonwoven fabric having Ag ion obtains best effect in disinfection.

The preferred embodiments herein disclosed are not intended to unnecessarily limit the scope of the invention. Therefore, simple modifications or variations belonging to the equivalent of the scope of the claims and the instructions disclosed herein for a patent are all within the scope of this invention.

What is claimed is:

1. The chitosan based dressing, comprising:
(a) a substrate;
(b) a layer of acrylic acid and N-isopropyl acrylamide (NIPAAm), said acrylic acid and said NIPAAm grafted and fixed on said substrate through a radiation; and
(c) a layer of chitosan, said chitosan grafted and fixed on said layer of acrylic acid together with NIPAAm through a radiation.

2. The dressing according to claim 1, wherein said substrate is a nonwoven fabric selected from a group consisting of polyethylene, polypropylene, polyethylene terephthalate (PET) and cotton.

3. The dressing according to claim 1, wherein said substrate is made of polyvinyl chloride (PVC).

4. The dressing according to claim 1, wherein said substrate is made of a material in a form selected from a group consisting of a film form and a flat form, said material selected from a group consisting of macromolecule and cellulose.

5. The dressing according to claim 1,
wherein said layer of acrylic acid together with NIPAAm is a layer of a similar monomer of vinyl monomer; and
wherein said similar monomer of vinyl monomer is selected from a group consisting of acrylic acid, NIPAAm with derivatives thereof, 2-hydroxy ethyl methacrylate, 2-hydroxy ethyl methacrylate with derivatives thereof, vinylpyridine, and vinylpyridine with derivatives thereof.

6. The dressing according to claim 1, wherein said layer of acrylic acid together with NIPAAm is a layer of hydrogel selected from a group consisting of acrylic acid and 2-hydroxy ethyl methacrylate.

7. The dressing according to claim 1, wherein a mixture rate of said acrylic acid and said NIPAAm is selected from a group consisting of 1:0, 1:1 and 3:1.

8. The dressing according to claim 1, wherein a thickness of said chitosan is between 1 wt % and 5 wt % with a 0.1M solution of acetic acid.

9. The dressing according to claim 1, wherein said chitosan is grafted as a polymer selected from a group consisting of a disordered copolymer and a dual-grafted polymer.

10. The dressing according to claim 1, wherein a thickness of said of acrylic acid together with NIPAAm is smaller than 50%.

11. The dressing according to claim 10,
wherein said of acrylic acid together with NIPAAm contains a photo initiator with a thickness smaller than 5%; and
wherein said photo initiator is selected from a group consisting of benzoin ethyl ether, phenyl benzoyl methanol, diphenylglyoxal, benzoyl peroxide, azobisisobutyro and tert-butyl hydoperoxide.

12. The dressing according to claim 1, wherein said radiation is selected from a group consisting of an ionizing radiation and an UV (ultraviolet) radiation.

13. The dressing according to claim 12, wherein a duration of said ionizing radiation is 10 to 100 minutes.

14. The dressing according to claim 1,
wherein said dressing with chitosan contains metallic ion for disinfecting; and
wherein said metallic ion is selected from a group consisting of Ag ion, Zn ion and Al ion.

15. The dressing according to claim 1, wherein said dressing with chitosan contains a surface of a dense porous structure.

16. The dressing according to claim 1, wherein said dressing with chitosan is an dressing with antisepsis, hydrotaxis and temperature-sensitivity.

17. The dressing according to claim 1,
wherein said layer of acrylic acid together with NIPAAm further comprises:
(a) a layer of acrylic acid, said acrylic acid grafted and fixed on said substrate through a radiation,
(b) a layer of peroxyacid group, said peroxyacid group formed by sulfuric acid and hydrogen peroxide through an peracid process, and
(c) a layer of acrylic acid together with NIPAAm, said acrylic acid and said NIPAAm grafted and fixed on said layer of peroxyacid group through a radiation; and
wherein said chitosan of said layer of chitosan is grafted and fixed on said layer of acrylic acid together with NIPAAm by a freeze-drying process.

18. The dressing according to claim 17,
wherein said radiation is an ionizing radiation of gamma ray; and
wherein a radiation amount of said ionizing radiation is between 5 kGy to 60 kGy through a duration of 0.5 to 10 hours.

19. The dressing according to claim 17, wherein said peracid process has a duration of 0.5 hour to 6 hours with a mixture of sulfuric acid and hydrogen peroxide ended with a washing of a cleaning agent of methanol afterward.

20. The dressing according to claim 17, wherein a temperature of sa id freeze-drying process is between 0° C. and −196° C.
